(19) United States
(12) Patent Application Publication
(10) Pub. No.: US 2011/0184454 A1
(43) Pub. Date: Jul. 28, 2011

(54) EMBOLIC IMPLANTS

(75) Inventors: David BARRY, Livermore, CA (US); Ben Tompkins, Danville, CA (US); Delilah Hui, American Canyon, CA (US); Arthur John Lockhart, San Ramon, CA (US)

(73) Assignee: Penumbra, Inc., Alameda, CA (US)

(21) Appl. No.: 12/695,035

(22) Filed: Jan. 27, 2010

(51) Int. Cl. A61F 2/01; B23P 11/00

(52) U.S. Cl. 606/200; 29/428

(57) ABSTRACT

Embolic coils are disclosed. The coils include an inner coil, an outer coil, and a stretch resistant member. Some embodiments include a large diameter outer coil formed from a small diameter wire. The inner coils may be either closed pitch or open pitch. Alternative coils include an inner coil that is shape set to a diameter that is larger than the diameter of the outer coil. Another alternative coil has multiple stretch resistant members.

### FIELD OF THE INVENTION

The present invention relates generally to the fields of intravascular implant devices, and more specifically to embolic coils.

### BACKGROUND

Embolization is a commonly practiced technique for treatment of brain aneurysm, arterio-venous malformation, tumors, and other conditions for which vessel occlusion is a desired treatment option. A typical occlusion coil is a wire coil having an elongate primary shape with windings coiled around a longitudinal axis. In the embolization procedure for treatment of aneurysm, a catheter is introduced into the femoral artery and navigated through the vascular system under fluoroscopic visualization. The coil in the primary shape is positioned within the catheter. The catheter distal end is positioned at the site of an aneurysm within the brain. The coil is passed from the catheter into the aneurysm. Once released from the catheter, the coil assumes a secondary shape selected to optimize filling of the aneurysm cavity. Multiple coils may be introduced into a single aneurysm cavity for optimal filling of the cavity, and costs typically increase with the number of coils required and the length of time required to successfully complete a procedure.

Proper positioning and anchoring of the coils is vital to a successful procedure. The deployed coils serve to block blood flow into the aneurysm and reinforce the aneurysm against rupture, while obstruction of blood flow through the healthy vessel must be avoided. Occasionally, repositioning of one or more coils is required during a procedure. Accordingly, an implant must be readily retractable within the catheter for repositioning. If one or more coils are not readily retractable during a procedure, an increase in the length of time required to complete a procedure may result. And most undesirably, a coil that requires repositioning but is not readily retractable into the catheter may prevent completion of a successful procedure entirely.

One type of coil is formed of a wire coiled to have a primary coil diameter. Additionally, such a coil may have a stretch resistant member enclosed by the coiled wire and anchored to one or both ends of the coil, or unattached and “free-floating” within the central lumen of the coil. The stretch resistant member may be shape set to impart a secondary shape to the coil, which the coil resumes within an aneurysm cavity or other treatment site. In order to facilitate dense filling or packing of a coil or coils within an aneurysm, and in order to decrease the length of time required to perform a procedure, a large diameter primary coil formed from a small diameter wire may be desired. However, a large primary diameter coil formed from a small diameter wire may be easily plastically deformed. Further, the individual adjacent turns and the pitch of such a coil are prone to shifting, especially if the wire has undergone deformation. Such a shift is further likely to cause difficulties in retrieval of a coil upon retraction of the coil into a catheter. Specifically, adjacent turns of a coil are likely to “catch” on the edge of the catheter during retraction, causing undesirable resistance and generally prevent a smooth retraction, or preventing retraction entirely.

It is desirable to avoid deformation of the wire when packing a coil into tight bends. Further, it is desirable that a coil be easily retrievable from the vessel. Accordingly, it is an object of the invention to provide a relatively soft, large diameter coil that will fill and expeditiously pack a treatment site densely. It is a further object of the invention to provide a coil that will not easily deform, undergo a shift in coil pitch, and/or catch on the catheter during retrieval of the coil.

### BRIEF DESCRIPTION OF THE DRAWINGS

FIG. 1 is a side elevation view of a prior art embolic coil.

FIG. 2 is a side elevation view of a prior art embolic coil in a partial cross section, during retraction of the coil into the distal end of an implant tool.

FIG. 3 is a side elevation view of an embolic coil according to the invention during retraction of the coil into the distal end of an implant tool.

FIG. 4 is a cross sectional view of an embolic implant according to the invention.

FIG. 5 is a cross sectional view of an alternative embodiment according to the invention.

FIG. 6 is a cross sectional view of another alternative embodiment according to the invention

FIG. 7 is a side elevation view of yet another alternative embodiment according to the invention, shown in partial cross section.

### DETAILED DESCRIPTION

Referring to FIG. 1, an example of a prior art coil that has undergone permanent plastic deformation is illustrated. A coil formed of a small diameter wire and having a large diameter primary shape may be desirable for filling and packing an aneurysm, but it is susceptible to plastic deformation during both intravascular delivery and packing/filling of an aneurysm. Such a coil is not readily retractable into a catheter.

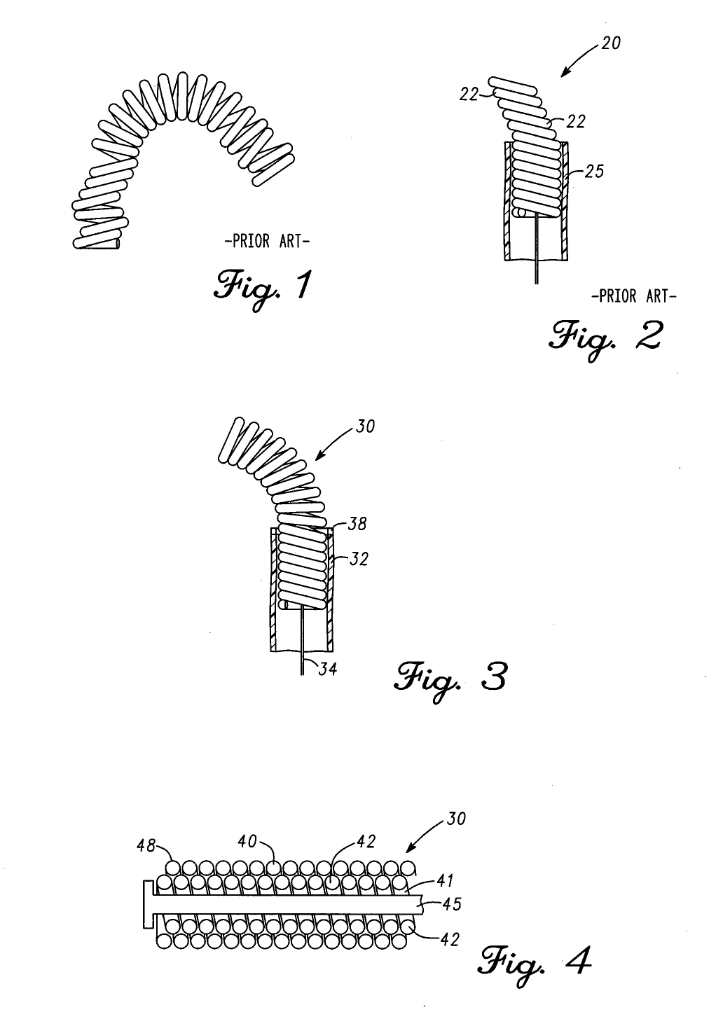

FIG. 2 illustrates an example of a prior art coil and one of its shortcomings. Prior art coil 20 is shown during attempted retraction of coil 20 into the distal end of catheter 25. Prior art coil 20 is formed of a series of adjacent windings 22. Adjacent windings 22, which had previously emerged from the distal end of catheter 25, have shifted with respect to one another. Consequently, upon attempts to retract coil 20 into the distal end of catheter 25, some of windings 22 have caught on the edge of catheter 25. A smooth retraction of coil 20 will thereby be prevented, and may potentially prevent retraction entirely. Consequently, prior art coil 20 is undesirable for use in treatment.

FIG. 3 illustrates the contrast of retraction of an implant according to the invention. In FIG. 3, implant 30 is undergoing retraction into the distal end of microcatheter 32 which may be similar to the catheter 25 used in the above example. Specifically, microcatheter 32 is an elongate flexible catheter proportioned to be received within the lumen of a corresponding guide catheter and advanced beyond the distal end of the guide catheter to the cerebral vasculature where an aneurysm to be treated is located. Suitable dimensions for the microcatheter include inner diameters of 0.010″ to 0.045″, outer diameters of 0.024″ to 0.056″, and lengths from 75 cm to 175 cm. One preferred embodiment utilizes the following dimensions: 0.025 in ID, 0.039 in Distal OD (3F), 0.045 in Proximal OD (3.5F), and length of 145-155 cm. Marker bands 38 facilitate fluoroscopic visualization of the microcatheter position during the course of an implantation procedure. Microcatheter 32 includes a lumen proportioned to receive the embolic implant 30 and the shaft of the insertion tool 34. Where the implant is within the lumen of the microcatheter, the surrounding lumen walls restrain the coil in the generally elongated shape shown in FIG. 3. As the implant exits the microcatheter, the implant assumes its secondary shape. The distal most end of implant 30 is shown partially in such a secondary shape. Following release of implant 30, microcatheter 32 is withdrawn from the vessel.

The contrast in ease of retraction of implant 30 is a result of the construction of the implant itself. Details of the embolic implant 30 are shown in cross section in FIG. 4. Implant 30 is formed of a wire 38 coiled to form outer coil 40 defining lumen 32 there through, and of a primary coil diameter D1 of approximately 0.018-0.045 inches, although smaller or larger diameters may instead be used. The pitch of outer coil 40 may be uniform as shown, or it may vary along the length of the coil, or different sections of the coil may be formed to have different pitches. The wire material selected for outer coil 40 is preferably one capable of fluoroscopic visualization, such as Platinum/Iridium, Platinum/Tungsten, or other suitable material. Alternatively, outer coil 40 may be formed of NiTi or stainless steel, rendering outer coil 40 less susceptible to permanent plastic deformation. In one embodiment, the wire forming outer coil 40 has a diameter of approximately 0.0020 inches or less.

Implant 30 also includes inner coil 42 which may be formed in the same manner and of the same material as outer coil 40, or of a different material. If, for example, outer coil 40 is formed of NiTi or stainless steel, inner coil 42 may be formed of platinum or other suitable material known to confer radiopacity on implant 30. Although other configurations are possible, in the example of FIG. 4, inner coil 42 is unattached, or “floating” within lumen 32, and its windings are of a relatively closed pitch. Inner coil 42 helps to maintain the pitch of outer coil 40 when the coil is placed under tension and/or pressure. During implantation, inner coil 42 helps in repositioning of the implant (if needed). Inner coil 42 makes the implant easier to retract, and maintain close positioning of coil windings during manipulation and retraction of the implant. Further, during use, inner coil 42 prevents a shift in pitch of the windings of outer coil 40 during retraction of implant 30 into a catheter (not shown). Accordingly, during a typical procedure, implant 30 can be retracted and repositioned more readily than a prior art coil.

Implant 30 further includes stretch resistant wire or member 45, manufactured from NiTi or other suitable shape memory material, and disposed within lumen 32. Implant 30 will have a secondary three-dimensional shape when released to a treatment site (not shown). The secondary shape can be helical, spherical, multi-lobal or any other shape desired to fill the aneurysm void. In this example, stretch resistant member 45 is the element that confers the secondary shape upon implant 30. The process for imparting this shape is to temperature set the stretch resistant member 45 into the desired shape. Stretch-resistant member 45 can be in a diameter range of 0.0005″ to 0.003″ or greater.

The stretch resistant wire prevents the inner coil 42 and outer coil 40 from stretching when deployed, repositioned, or withdrawn from the aneurysm. This stretch resistant wire will not yield when placed in tension during repositioning. Conversely, stretch resistant wire will prevent compaction of adjacent coils, likely improving long term performance of implant 30 following implantation. Stretch resistant wire 45 will have a yield strength approximately 0.5 lbs. In a preferred embodiment, the stretch resistant wire is shape set to give the embolic implant 30 its predetermined secondary shape. In alternative embodiments, outer coil 40, and/or inner coil 42, or both coils and the wire may be shape set to give the implant 30 its secondary shape.

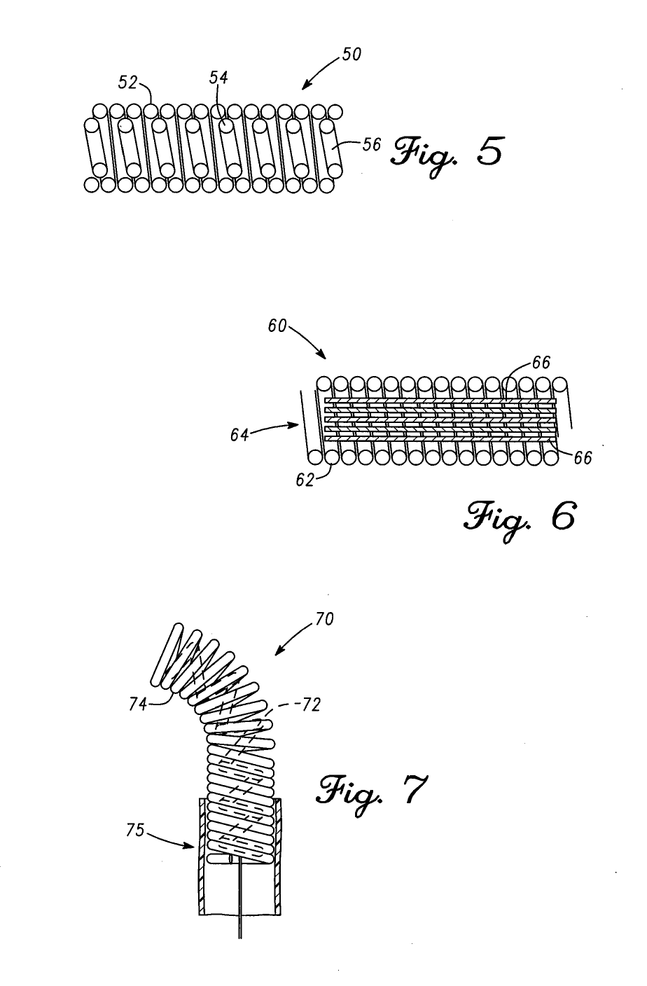

An alternative embodiment according to the invention is illustrated in FIG. 5. Using some suitable combination of the materials described in relation to FIG. 4 above, implant 50 is formed of outer coil 52 and inner coil 54. Although not included in the embodiment illustrated in FIG. 5, implant 50 may also include a stretch resistant member similar to that described in relation to FIG. 4 above. In the example of FIG. 5, inner coil 54 is formed of open pitch windings, and disposed within lumen 56 of first coil 52. Inner coil 54 confers many of the same advantages upon implant 50 as that described above in relation to FIGS. 3 and 4. Alternatively, inner coil 54 may be additionally constructed to exert an outward radial pressure within lumen 56 of first coil 52. Inner coil 54 may comprise a larger diameter than the inner diameter of first coil 52 prior to its loading into lumen 56. Alternatively, inner coil 54 may be shape set to a larger diameter than outer coil 52. Additional alternative means for imparting an outward radial force to inner coil 54 may be suitable and within the scope of the invention.

FIG. 6 is a cross sectional view of another alternative embodiment according to the invention. In the embodiment of FIG. 6, implant 60 is formed of coil 62 manufactured from suitable materials as described above in relation to FIGS. 3-5 and defining lumen 64. In this example, implant 60 further comprises a plurality of stretch resistant members 66. Stretch resistant members 66 may be formed from NiTi or other suitable material. Stretch resistant members 66 have relatively small diameters, and confer stability on coil 62 without sacrificing the needed flexibility of implant 60. Stretch resistant members 66 also prevent a shift in pitch of the windings of coil 62 before and/or during retraction of implant 60 into a catheter. Accordingly, implant 60 is more readily retracted and repositioned than a prior art coil.

FIG. 7 is a side elevation view of yet another alternative embodiment according to the invention, shown in partial cross section. Implant 70 is illustrated within the distal end of catheter 75. Implant 70 will resume its secondary shape when it is released from catheter 75. Implant 70 is formed from inner coil 72, constructed of a suitable material such as Platinum. Inner coil 72 may be susceptible to plastic deformation during delivery and deployment. However, implant 70 further comprises outer coil 74. Although other materials and configurations may be suitable within the scope of the invention, in this example, outer coil 74 is constructed of NiTi wound with an open pitch. During retraction of implant 70 into the distal end of catheter 75, outer coil 74 will serve as a guide rail between catheter 75 and implant 70. Accordingly, no friction will be felt between the foregoing surfaces during retraction of the device, and during a procedure, implant 70 will be more readily repositioned than a prior art coil.

The foregoing description provides examples of embodiments to facilitate explanation of the invention, and those embodiments can be varied within the scope of the invention. The foregoing descriptions are not intended as limitations of the invention herein.

We claim:

1. An embolic implant comprising a first coil having a lumen extending through a substantial length of said coil, and a second coil extending through a substantial portion of said lumen.

2. The implant according to claim 1 wherein said second coil comprises a second coil lumen extending there through, and said implant further comprises a member extending through a substantial portion of said second coil lumen.

3. The implant according to claim 1 wherein said first coil comprises a substantially closed pitch second coil comprises a substantially closed pitch.

4. The implant according to claim 1 wherein said first coil comprises a substantially closed pitch second coil comprises a substantially open pitch.

5. The implant according to claim 1 wherein said first coil comprises stainless steel and said second coil comprises Platinum.

6. The implant according to claim 1 wherein said first coil comprises NiTi and said second coil comprises Platinum.

7. The implant according to claim 2 wherein said member comprises NiTi.

8. The implant according to claim 1 where said first coil comprises a substantially open pitch and second coil comprises a substantially closed pitch.

9. The implant according to claim 8 where said first coil comprises NiTi and said second coil comprises Platinum.

10. The implant according to claim 1 wherein said second coil exhibits an outward radial force.

11. The implant according to claim 2 wherein said implant comprises a primary configuration and a secondary configuration, and wherein said member confers said secondary configuration upon said implant.

12. The implant according to claim 1 further comprising an outer diameter and formed from a wire having a wire diameter, wherein the ratio of said outer diameter to said wire diameter is greater than 10.

13. A method of manufacture of an embolic implant comprising an implant central lumen, the method comprising the steps of:
providing a first coil comprising a first coil lumen;
providing a second coil;
disposing said second coil within a substantial portion of the first coil lumen.

14. The method of claim 13 further comprising the step of disposing a member within said central implant lumen.

15. The method of claim 13 where said first coil comprises a closed pitch and said second coil comprises a closed pitch.

16. The method of claim 13 where said first coil comprises a closed pitch and said second coil comprises an open pitch.

17. The method of claim 13 where said first coil comprises an open pitch and said second coil comprises a closed pitch.

18. The method of claim 13 where said first coil is formed from a wire comprising a wire diameter, and said first coil comprises an outer diameter, wherein the ratio of said outer diameter to said wire diameter is greater than 10.

19. A method of manufacturing an embolic implant, the method comprising the steps of:
providing a coil comprising a lumen extending through a substantial length of said coil;
disposing a plurality of members within said lumen.

20. An embolic implant comprising a coil having a lumen and a plurality of members disposed within said lumen.
